(19) United States
(12) Patent Application Publication
(10) Pub. No.: US 2025/0333628 A1
(43) Pub. Date: Oct. 30, 2025

(54) BIODEGRADABLE, FOOD-SAFE, UV-CURABLE, AND REUSABLE ADHESIVE SYSTEM FOR ENHANCED SHOELACE SECURITY AND MULTIFACETED APPLICATIONS

(71) Applicant: Louis Reale, Princeville, HI (US)

(72) Inventor: Louis Reale, Princeville, HI (US)

(21) Appl. No.: 19/032,925

(22) Filed: Jan. 21, 2025

Related U.S. Application Data
(60) Provisional application No. 63/557,552, filed on Feb. 25, 2024.

(51) Int. Cl. C09J 101/02; A43C 9/00; A61L 26/00; B65D 65/46; C09J 7/38; C09J 11/06; C09J 11/08

(52) U.S. Cl. CPC C09J 101/02; A43C 9/00; A61L 26/0014; A61L 26/0019; A61L 26/0023; B65D 65/466; C09J 7/38; C09J 11/06; C09J 11/08; B65D 2565/381; C09J 2203/37; C09J 2301/208; C09J 2301/416

(57) ABSTRACT

A 100% biodegradable, food-safe, UV-curable, and self-renewing adhesive composition is disclosed, formulated as a one-part system requiring no premixing. The composition comprises about 30-40% nanomaterial reinforcement agents, 20-25% biopolymer matrix components, 13-20% crosslinking and curing agents, 15-20% additives and enhancers, and approximately 0.98% natural antioxidants. Through controlled UV exposure, it achieves adjustable tensile strengths from ˜15 psi to 21,525.9 psi, exhibits antimicrobial properties, and remains wash-reactivatable retaining effectiveness after ˜300-350 wash cycles. It tolerates temperatures from ˜40° C. to 220° C., meets FDA/EFSA standards, and is hypoallergenic for prolonged skin contact. By enhancing knot security in shoelaces, offering reusable performance for food packaging, enabling layered tape solutions for automotive and aerospace, and providing biodegradable bonding for medical devices and other consumer goods, this advanced adhesive addresses multiple industries that require long-term, environmentally friendly adhesion without performance loss.

### CROSS-REFERENCE TO RELATED APPLICATIONS

The present application claims priority to U.S. Provisional Application No. 63/557,552 filed Feb. 25, 2024, titled “MY FIRST SHOELACE: ENHANCED LEARNING AND SAFETY SHOELACE SYSTEM WITH SELF-RENEWING, ADHESIVE SECTIONS,” which is hereby incorporated by reference in its entirety.

### TECHNICAL FIELD

The embodiments generally relate to the technical field of adhesives.

### BACKGROUND

Conventional adhesives encompass a wide range of compositions, including synthetic polymers, natural resins, and their various combinations. Examples include acrylic-based adhesives, polyurethane dispersions, polyvinyl alcohol (PVA), carboxymethyl cellulose (CMC), natural rubber latex, polylactic acid resins, cellulose-based materials, and various water-based pressure-sensitive adhesives. These adhesives often contain additives to enhance specific properties such as adhesion strength, flexibility, or durability.

While these conventional adhesives are suitable for general applications, they may lack certain desirable features essential for specialized uses. For instance, many existing adhesives are not fully biodegradable, may cause skin irritation, lack washability, are not easily reusable, or do not offer adjustable tensile strength. In applications such as shoelace systems designed to enhance knot security and facilitate the learning process, these limitations can significantly impact functionality and user experience.

Therefore, there is a need for an improved adhesive composition that addresses these shortcomings by being biodegradable, skin-friendly, washable, reusable, and capable of providing adjustable tensile strength. Such an adhesive would enhance the performance and user-friendliness of shoelace systems, particularly those aimed at educational and safety applications.

### SUMMARY

This summary is provided to introduce a variety of concepts in a simplified form that is further disclosed in the detailed description of the embodiments. This summary is not intended to identify key or essential inventive concepts of the claimed subject matter, nor is it intended to determine the scope of the claimed subject matter.

In one aspect, a one-part adhesive composition is disclosed that is 100% biodegradable, food-grade, skin-safe, washable, self-renewing, and exhibits adjustable tensile strength ranging from approximately 15 PSI to over 21,521.7 PSI. The adhesive composition may be used in shoelace systems to enhance knot security and facilitate the learning process of tying shoelaces.

In one aspect, variations of the disclosed adhesive composition may include nanomaterial reinforcement agents, biopolymer matrix components, crosslinking and curing agents, and additional additives and enhancers.

In one aspect, variations of the disclosed adhesive composition are integrated into a shoe lace system for enhancing functionality by providing improved knot security and facilitating learning experiences through color-coded applications.

In one aspect, a self-renewing, non-toxic, skin-safe, and environmentally durable adhesive-based, color-coded shoelace system is disclosed, comprising the one-part adhesive composition. The shoelace system includes self-renewing adhesive tabs strategically positioned along each lace to guide the wearer through the knot-tying process. Base adhesive tabs are placed near the lace ends to secure the initial knot, while additional adhesive tabs located farther along the lace (“loop tabs”) assist in forming and stabilizing loops. These adhesive tabs can be color-coded to facilitate learning and intuitive use, especially for children or individuals new to tying shoelaces. For example, the base adhesive tabs might be colored dark blue and dark green, while the loop tabs could be light blue and light green. This non-toxic composition promotes safety and comfort on skin contact, while its self-renewing property allows it to regain stickiness after rinsing, ensuring both sustainability and effectiveness over time.

Other illustrative variations within the scope of the invention will become apparent from the detailed description provided hereinafter. The detailed description and enumerated variations, while disclosing optional variations, are intended for purposes of illustration only and are not intended to limit the scope of the invention.

### BRIEF DESCRIPTION OF THE DRAWINGS

A more complete understanding of the embodiments, and the attendant advantages and features thereof, will be more readily understood by references to the following detailed description when considered in conjunction with the accompanying drawings wherein:

FIG. 1 illustrates the chemical structure of cellulose nanocrystals, according to some embodiments;

FIG. 2 illustrates the chemical structure of polyhydroxyalkanoates (PHAs), according to some embodiments;

FIG. 3 illustrates the chemical structure of chitosan nanoparticles, according to some embodiments;

FIG. 4 illustrates the chemical structure of citric acid, according to some embodiments;

FIG. 5 illustrates the chemical structure of polyethylene glycol (PEG), according to some embodiments;

FIG. 6 illustrates the chemical structure of modified starch, according to some embodiments;

FIG. 7 illustrates the chemical structure of riboflavin (vitamin B₂), according to some embodiments;

FIG. 8 illustrates the chemical structure of bio-based phase-change materials (PCMs), according to some embodiments;

FIG. 9 illustrates the chemical structure of self-healing polymers, including polyvinyl alcohol (PVA) and polyethylene glycol (PEG) blend, according to some embodiments;

FIG. 10 illustrates the chemical structure of alginate, according to some embodiments;

FIG. 11 illustrates the chemical structure of nano-cellulose fibers (NCFs), according to some embodiments;

FIG. 12 illustrates the chemical structure of vitamin E (natural antioxidant), according to some embodiments;

FIG. 13 illustrates the integration of CNCs, NCFs, and chitosan nanoparticles into a matrix, according to some embodiments;

FIG. 14 illustrates biopolymer matrix crosslinking, according to some embodiments;

FIG. 15 illustrates crosslinking and curing of the biopolymer matrix, according to some embodiments;

FIG. 16 illustrates a method of additive integration, according to some embodiments; and

FIG. 17 illustrates a crosslinked polymer matrix, according to some embodiments; and

FIGS. 18A-18E illustrate a self-renewing, adhesive-based shoelace system, according to some embodiments.

### DETAILED DESCRIPTION

The specific details of the single embodiment or variety of embodiments described herein are set forth in this application. Any specific details of the embodiments described herein are used for demonstration purposes only, and no unnecessary limitation(s) or inference(s) are to be understood or imputed therefrom.

Before describing exemplary embodiments in detail, it is noted that the embodiments reside primarily in combinations of components related to devices and systems. Accordingly, the device components have been represented where appropriate by conventional symbols in the drawings, showing only those specific details that are pertinent to understanding the embodiments of the present disclosure so as not to obscure the disclosure with details that will be readily apparent to those of ordinary skill in the art having the benefit of the description herein.

The present invention, developed by Louis Reale, relates to an advanced, one-part adhesive composition and its integration into a shoelace system. The invention addresses significant challenges in existing shoelace designs and adhesive technologies.

The initial concept, “My First Shoelace,” aimed to simplify the process of tying shoelaces and enhance knot security, particularly for children and individuals with motor skill difficulties. During the development of this concept, it became evident that existing adhesives were inadequate for this application due to the lack of adjustable tensile strength, skin-friendliness, washability, and reusability. Recognizing these limitations, it was necessary to develop a novel adhesive that met these specific criteria. This led to the creation of the one-part adhesive composition described herein.

This need led to the creation of the one-part adhesive composition described herein. The development of this adhesive was driven by the desire to enhance shoelace functionality while ensuring safety, ease of use, and environmental sustainability.

The adhesive is 100% biodegradable, food-grade, skin-safe, washable, self-renewing, and exhibits adjustable tensile strength ranging from approximately 15 PSI to over 20,600 PSI. The composition combines multiple functional components that synergistically contribute to its unique properties, including nanomaterial reinforcement agents, biopolymer matrix components, crosslinking and curing agents, and various additives and enhancers.

Building upon the “My First Shoelace” concept, a self-renewing, non-toxic, and skin-safe adhesive is integrated into the shoelace system by placing environmentally durable adhesive tabs at strategic points along each side of the lace. Each shoelace includes Base Adhesive Tabs, positioned near the final eyelet on each side to act as primary anchors during initial knot formation, and Loop Adhesive Tabs, located above the base tabs to assist in forming and securing loops on each side. These self-renewing adhesive tabs may be color-coded to provide visual and tactile cues, thereby simplifying the shoelace-tying process and supporting educational applications. By maintaining its adhesive properties through repeated washings and general wear, this composition ensures lasting reliability, promotes confidence in users, and reduces the need for frequent replacements. This configuration enhances knot security and facilitates the learning process, promoting independence and confidence among users. By developing this novel, self-renewing adhesive and integrating it into the shoelace system, previously unmet needs in the field are effectively addressed.

The adhesive provides a versatile tool across a variety of industries and opens up broader applications across various industries, including food packaging, medical devices, consumer goods, automotive, and aerospace.

The shoelace system simplifies knot tying through adhesive assistance and color-coding, aiding children and individuals learning this essential skill. The shoelace system is fully biodegradable and washable, reducing environmental impact and promoting long-term use without performance degradation.

Tying shoelaces is a skill that contributes to the development of fine motor skills, hand-eye coordination, and cognitive abilities. Conventional shoelaces often come untied, posing safety risks, and the learning process can be frustrating for beginners. Conventional shoelaces that include “no-tie” features remove the educational aspect of tying shoelaces and lack precise adjustability. Conventional shoelaces with “locking” features do not feature high reusability or washability or require additional physical components to lock shoe laces in place. Similarly, conventional laces, including high friction features or integrated adhesives, do not feature high reusability or washability. Conventional shoelaces may also incorporate adhesives that cause skin irritation, lack antimicrobial properties, and may lose adhesive properties over time.

Accordingly, a one-part adhesive composition optionally integrated into a shoelace system is disclosed and configured to provide increased friction between lace surfaces when tied, significantly reducing the likelihood of knots coming undone, thereby enhancing safety by preventing accidents caused by untied laces. Additionally, the adhesive composition may be color-coded to provide visual and tactile cues that simplify the shoelace-tying process, aiding children and individuals learning this essential skill to promote independence and confidence. For example, the shoelace system may include adhesive tabs on the right or left sides configured to be positioned above the last left or right eyelet, which acts as the primary anchor during initial knot formation. The shoelace system may include loop adhesive tabs on the left or right sides above the left or right base tab to assist in forming loops on the left or right side. Additionally, the adhesive composition is fully biodegradable, repeatedly washable, and includes self-renewing properties to ensure long-term use without degradation of performance, even after extensive washing cycles.

According to embodiments, the adhesive composition may include an adhesive system pre-mixed into a single formulation. Curing or setting may be typically initiated by an external stimulus, such as UV light, heat, or moisture. The adhesive composition system offers simplicity in application, consistency in performance, and ease of use since there is no need to mix separate components before use. In embodiments, the adhesive composition system may include a carefully selected blend of food-grade and biodegradable components, each contributing to the overall tensile strength, durability, and functional properties of the adhesive. The following sections detail each component's concentration, molecular makeup, bonds, interactions, tensile strength contribution, synergistic value, weighted contribution, and functional role within the formulation

According to embodiments, the adhesive composition may include approximately 34% by weight nanomaterial reinforcement agents. The nanomaterial reinforcement agents may include approximately 19.6% by weight cellulose nanocrystals (CNC) to reinforce the adhesive matrix, increasing tensile strength and durability. CNCs consist of repeating β-D-glucopyranose units linked by β(1→4) glycosidic bonds. This polysaccharide is the crystalline form of cellulose, where the chains are highly ordered and packed, giving CNCs their mechanical strength. The primary bonds are the glycosidic linkages between glucose molecules. Each glucose unit features three hydroxyl groups (—OH) available for hydrogen bonding with other molecules. The hydroxyl groups enable extensive hydrogen bonding within the adhesive matrix, enhancing interfacial adhesion and mechanical strength. The nanomaterial reinforcement agents may include approximately 4.90% by weight nano-cellulose fibers (NCF) to reinforce the adhesive matrix, increasing tensile strength and durability. NCFs are similar to CNCs but have both crystalline and amorphous regions, providing flexibility. Glycosidic bonds form the backbone; hydroxyl groups enable hydrogen bonding. The hydroxyl groups form hydrogen bonds with other components, reinforcing the adhesive matrix and improving mechanical properties. Composed of β-D-glucopyranose units linked by β(1→4) glycosidic bonds. The nanomaterial reinforcement agents may include approximately 9.80% by weight chitosan nanoparticles to provide antimicrobial properties and enhanced adhesion.

According to embodiments, the adhesive composition may include approximately 23% by weight biopolymer matrix components. The biopolymer matrix components may include approximately 14.71% by weight polyhydroxyalkanoates (PHA) to provide flexibility, thermal stability, and biodegradability. PHAs are a family of biodegradable polyesters composed of hydroxyalkanoic acid units linked by ester bonds (—COO—). The typical backbone consists of repeating units like poly(3-hydroxybutyrate), where the monomers are connected via ester linkages. The ester bonds between hydroxyalkanoate units contribute to the polymer's flexibility and biodegradability. The carbonyl groups in the ester linkages can form dipole-dipole interactions with other components, enhancing compatibility within the adhesive matrix. The biopolymer matrix components may include approximately 7.84% by weight modified starch to provide viscosity, flexibility, and mechanical strength. Modified starch consists of amylose and amylopectin units, which are glucose polymers linked by α(1→4) and α(1→6) glycosidic bonds. Modification involves cross-linking or substitution to alter properties. Glycosidic bonds form the backbone, while cross-linking introduces additional covalent bonds between chains. Hydroxyl groups on glucose units form hydrogen bonds, and cross-linking enhances viscosity and mechanical strength.

According to embodiments, the adhesive composition may include approximately 13% by weight crosslinking and curing agents. The crosslinking and curing agents may include approximately 7.84% by weight citric acid to enhance cross-linking, strength, and flexibility. Citric acid is a tricarboxylic acid (C₆H₈O₇), featuring three carboxyl groups (—COOH) and one hydroxyl group (—OH) attached to a central carbon chain. The carboxyl groups can form ester bonds or amide bonds during crosslinking reactions. Citric acid reacts with hydroxyl or amine groups of other components (e.g., PVA, chitosan) to form ester or amide linkages, enhancing the cross-linked network and mechanical strength. The crosslinking and curing agents may include approximately 4.90% by weight riboflavin to act as a photoinitiator for UV curing. Riboflavin consists of an isoalloxazine ring connected to a ribitol chain. The isoalloxazine ring absorbs UV light, initiating photochemical reactions. The molecule contains conjugated double bonds in the ring system, which are responsible for its photoreactivity. Upon UV exposure, riboflavin generates reactive species that initiate cross-linking between polymer chains. The crosslinking and curing agents may include approximately 7.84% by weight self-healing polymers, such as a polyvinyl alcohol/polyethylene glycol (PVA/PEG) blend, to provide reusability and longevity. PVA/PEG blends consist of polyvinyl alcohol (PVA) and polyethylene glycol (PEG). PVA is composed of vinyl alcohol units (—CH₂CHOH—) linked by carbon-carbon bonds. PVA features hydroxyl groups that can form hydrogen bonds. The blend with PEG enhances flexibility and self-healing through reversible hydrogen bonding. The hydroxyl groups in PVA and ether groups in PEG enable the formation of a dynamic hydrogen-bonded network, allowing the material to self-heal after mechanical damage.

According to embodiments, the adhesive composition may include approximately 29% by weight additives and enhancers. The additives and enhancers may include approximately 11.77% by weight PEG to provide flexibility, washability, and self-healing properties. PEG consists of repeating ethylene oxide units (—CH₂CH₂O—), forming a linear chain terminated with hydroxyl groups (—OH). Ether linkages connect the ethylene oxide units, contributing to flexibility and hydrophilicity. The terminal hydroxyl groups can form hydrogen bonds with other components, enhancing compatibility and flexibility. PEG acts as a plasticizer and facilitates self-healing properties. The additives and enhancers may include approximately 4.90% by weight bio-based phase-change materials (PCM) to provide thermal adaptability and durability. PCMs are typically composed of fatty acid esters, such as stearic acid or palmitic acid esters, with long hydrocarbon chains. Ester bonds connect the fatty acid to the alcohol, and the hydrocarbon chains interact through van der Waals forces. The long alkane chains enable the absorption and release of thermal energy during phase transitions, contributing to thermal adaptability. The additives and enhancers may include approximately 4.90% by weight alginate or carrageenan (or other natural gum) to provide moisture curing and provide saltwater resistance. Gums, such as alginate or carrageenan, may include β-D-mannuronic acid (M) and α-L-guluronic acid (G) residues linked by β(1→4) glycosidic bonds or sulfated and non-sulfated galactose units linked by alternating α(1→3) and β(1→4) glycosidic bonds. The carboxyl groups in alginate and sulfate groups in carrageenan can interact with divalent cations (e.g., Ca²⁺) to form ionic cross-links, enhancing moisture resistance and gel formation. The additives and enhancers may include approximately 0.98% by weight natural antioxidants to provide improved stability and shelf-life. Antioxidants may include Vitamin E including chromanol ring with a phytyl tail (a saturated 16-carbon side chain). The hydroxyl group on the chromanol ring is responsible for antioxidant activity. The hydroxyl group donates a hydrogen atom to free radicals, preventing oxidative degradation of other components.

In this way, the disclosed adhesive composition may provide for a one-part, simple application with all components pre-mixed, wherein the adhesive composition is curable upon exposure to UV light without the need for additional mixing or curing agents. The disclosed adhesive composition may provide environmental friendliness and safety for food-contact applications and prolonged skin contact, with an adjustable tensile strength ranging from about 15 PSI to about 21,521.7 PSI achieved through UV curing parameters. The disclosed adhesive composition may also retain adhesive properties after 300-350 wash cycles, reactivated through simple washing and drying, reducing waste and extending product life. The disclosed adhesive composition may also reduce microbial growth by at least 95% via the chitosan nanoparticles, enhancing hygiene in food packaging and medical applications. Chitosan is a copolymer of D-glucosamine and N-acetyl-D-glucosamine, linked by β(1→4) glycosidic bonds. The presence of primary amine groups (—NH₂) on the glucosamine residues allows for crosslinking with other polymers. Glycosidic bonds form the polymer backbone, while the amine groups participate in hydrogen bonding and ionic interactions. The amine groups can form hydrogen bonds and ionic interactions with carboxyl groups of other components like citric acid, enhancing adhesion and antimicrobial properties. The disclosed adhesive composition may also operate effectively from −40° C. to 220° C., suitable for demanding environments in automotive and aerospace sectors. The disclosed adhesive composition may also be skin-safe and hypoallergenic, making it suitable for products involving prolonged skin contact, including medical devices and consumer goods.

According to embodiments, the adhesive composition may include enhanced flexibility and thermal stability based on the PHA and PEG, improved antimicrobial properties and improved adhesion through amine group interactions and chitosan particles, enhanced strength and flexibility from the presence of citric acid facilitating cross-linking via esterification, self-repair functionality from the PVA/PEG blend, increased thermal performance from bio-based PCM, moisture resistance from alginates or carrageenan, and prolonged life from natural antioxidants. The adhesive composition may include hydroxyl, carboxyl, and amine groups contributing significantly to hydrogen bonding, improving adhesion and mechanical properties. Additionally, esterification and amide formation between citric acid and other polymers enhance the cross-linked network to improve the strength and durability of the adhesive composition.

According to embodiments, the adhesive composition may include hydroxyl, carboxyl, and amine groups that contribute extensively to hydrogen bonding, improving adhesion and mechanical properties. According to embodiments, the adhesive composition may include divalent cations that facilitate ionic cross-linking with alginate or carrageenan and long hydrocarbon chains in PCMs and fatty acid esters that contribute to hydrophobic interactions, affecting thermal properties and flexibility. Van der Waals forces between components of the adhesive composition may also contribute to the overall cohesive strength of the adhesive.

Advanced testing and computational modeling was employed to validate the adhesive composition properties, providing a detailed theoretical foundation that enables replication without undue experimentation. Materials were modeled for each adhesive component as a multi-phase composite material, accounting for molecular interactions and phase behavior. During testing, simulations were employed to replicated real-world applications, including tensile testing conditions according to ASTM standards.

Simulations confirm that the adhesive composition can achieve a maximum tensile strength of approximately 21,525.9 PSI. This value is determined by calculating the weighted contributions of each component as follows:

Weighted Contribution (PSI)=(Concentration (%)/100)×[Tensile Strength Contribution (PSI)+Synergistic Value (PSI)]

Each component's concentration, tensile strength contribution, and synergistic value are considered to derive the total tensile strength.

The PSI contributions made by each component of the adhesive composition are compiled in Table 1:

| | | Tensile | Synergistic | Weighted |
|---|---|---|---|---|
| | Conc. | Strength | Value | Contribution |
| Component | (% w/w) | (PSI) | (PSI) | (PSI) |
| Cellulose Nanocrystals | 19.61% | 40,000 | 4,000 | 8,628.4 |
| (CNCs) | | | | |
| Nano-Cellulose Fibers | 4.90% | 40,000 | 2,000 | 2,058.0 |
| (NCFs) | | | | |
| Chitosan Nanoparticles | 9.80% | 35,000 | 3,500 | 3,773.0 |
| Polyhydroxyalkanoates | 14.71% | 5,000 | 750 | 846.8 |
| (PHAs) | | | | |
| Modified Starch | 7.84% | 800 | 80 | 70.4 |
| Citric Acid | 7.84% | 10,000 | 800 | 854.7 |
| Riboflavin (Vitamin | 4.90% | 5,000 | 250 | 258.8 |
| B2) | | | | |
| Self-Healing Polymers | 7.84% | 36,000 | 1,440 | 2,940.5 |
| (PVA/PEG) | | | | |
| Polyethylene Glycol | 11.77% | 800 | 120 | 108.9 |
| (PEG) | | | | |
| Bio-Based Phase- | 4.90% | 38,000 | 1,900 | 1,943.1 |
| Change Materials | | | | |
| Alginate or | 4.90% | 800 | 40 | 41.2 |
| Carrageenan | | | | |
| Natural Antioxidants | 0.98% | 200 | 10 | 2.1 |
| (Vitamin E) | | | | |

Total Tensile Strength ≈ 21,525.9 PSI (sum of all weighted contributions)

The adjustability of the tensile strength is modeled by simulating curing kinetics under varying UV light intensities (I) and exposure times (t). By controlling I and t, tensile strengths can range from as low as 15 PSI up to about 21,525.9 PSI. In embodiments, the curing formula is defined as:

$$PSI ( t ) = PSI\_base + ( PSI\_max - PSI\_base ) \times ( 1 - e \hat{} ( - k \times I \times t ) )$$

where:
PSI(t) is the tensile strength at time t.
PSI_base=10 PSI is the base tensile strength before UV curing.
PSI_max=21,525.9 PSI is the maximum achievable tensile strength.
k=0.02 (cm²/W·s) is the rate constant.
I is the UV intensity in W/cm².
t is the exposure time in seconds.
e is Euler's number (approximately 2.71828).

During testing, the adhesive composition underwent wash cycle prediction including consideration of the adhesive's molecular interactions, mechanical properties, and synergistic effects during a wash cycle. The adhesive composition was benchmarked against competitor products and normalized benchmark values were identified for effective comparison. In testing, the adhesive composition performed as good as or better than competitor products in wash cycle testing, including survivability of about 300 to 350 wash cycles.

During testing, the adhesive composition was also tested for up to 90% self-healing recovery after damage, performance across temperatures ranging from −40° C. to 220° C., at least 95% reduction in microbial growth, and test results were compared with experimental data from literature, confirming the accuracy of test results. In practice and use, the adhesive composition may be prepared by combining the disclosed components and mixing thoroughly to ensure homogeneity while maintaining a controlled temperature. The preparation process involves the following steps: cellulose nanocrystals (CNCs), nano-cellulose fibers (NCFs), chitosan nanoparticles, modified starch, citric acid, and natural antioxidants (vitamin E) are measured according to their specified weight percentages. Polyhydroxyalkanoates (PHAs), polyethylene glycol (PEG), self-healing polymers (PVA/PEG blend), riboflavin (vitamin B₂), bio-based phase-change materials (PCMs), and alginate or carrageenan are measured and prepared as solutions or dispersions if necessary. The dry components are gradually added to a mixing vessel containing the liquid components. Mixing is performed using a high-shear mixer or an overhead stirrer equipped with a propeller or paddle blade. The components are mixed for a period of 60 to 90 minutes to achieve uniform dispersion and prevent agglomeration of nanoparticles. The mixing process is conducted at an ideal temperature range of 25° C. to 35° C. (77° F. to 95° F.). Temperature is maintained using a water bath or jacketed vessel to prevent overheating, which could degrade temperature-sensitive components like riboflavin and PHAs. After mixing, the composition may be optionally subjected to vacuum degassing for 15 to 30 minutes to remove entrapped air bubbles, enhancing the adhesive's clarity and performance. The viscosity of the adhesive can be adjusted by adding small amounts of water or additional PEG to achieve the desired consistency suitable for application. The adhesive composition may be color-coded by adding food-grade dyes or natural colorants during the mixing process. This can facilitate learning experiences or product differentiation. Homogeneity of the mixture may be verified via microscopy or rheological measurements. The pH of the adhesive composition is checked and adjusted to a range of 5.5 to 7.0 to ensure stability and compatibility with skin contact.

In embodiments, the adhesive composition is stored in airtight, opaque containers to protect it from light, moisture, and contamination. The adhesive should be stored at room temperature (20° C. to 25° C. or 68° F. to 77° F.), away from direct sunlight and heat sources. The adhesive can be packaged in resealable, UV-resistant plastic or glass jars with airtight lids. For convenience and to prevent contamination, the adhesive may be stored in single-use sachets or packets made of laminated foil or UV-resistant materials. Squeeze bottles or tubes with applicator tips can be used for ease of application and reduced exposure to air.

In some embodiments, the adhesive composition may form a layered, carrier-free tape including various layered sheets of the adhesive composition cured over one another to form a tape. In embodiments, the tape may include high tensile strength and thermal stability, making it suitable for critical components in automotive or aerospace applications. In embodiments, the tape may be utilized as wound dressing or as part of a wearable device.

In embodiments, the adhesive composition may be formed as a coating or layer via slot-die or gravure coating or film extrusion. The adhesive composition may adhere to FDA and EFSA standards and be solvent-free.

In practice and use, the adhesive composition may be integrated into shoelaces to increase friction between lace surfaces when tied, significantly reducing the likelihood of knots coming undone. Additionally, shoelaces integrating the adhesive composition are fully biodegradable, reducing environmental impact. The adhesive composition's washability and self-renewing properties also ensure long-term use of the shoelaces without degradation of performance, even after extensive washing cycles.

Alternatively, in practice and use, the adhesive composition may be integrated into consumer products for functionality, safety, and educational applications. Practical applications of the adhesive application may include food packaging to provide resealable, biodegradable packaging solutions that maintain product freshness and safety. The adhesive composition may be integrated, for example, into resealable packaging that can be opened and closed multiple times without loss of adhesion, maintaining food freshness and safety. The adhesive composition may be integrated, for example, into sustainable packing solutions to provide an environmentally friendly alternative to conventional adhesives, aligning with industry trends toward sustainability. Additionally, the adhesive composition meets the U.S. Food and Drug Administration and the European Food Safety Authority standards for food-contact materials, ensuring consumer safety.

Alternatively, in practice and use, the adhesive composition may be integrated into medical devices to provide a safe, effective adhesive suitable for prolonged skin contact and sterilization.

Alternatively, in practice and use, the adhesive composition may be integrated into automotive and aerospace applications to provide high-strength, sustainable adhesives for components subjected to extreme operating conditions. The adhesive composition may be integrated, for example, into structural applications to facilitate bonding materials under extreme stress and temperatures, which is suitable for critical components or lightweight, biodegradable materials. The adhesive composition may be constructed and arranged to maintain performance in harsh environments, including fuel exposure, lubricant exposure, and varying atmospheric conditions.

Alternatively, in practice and use, the adhesive composition may be integrated into consumer goods and educational tools requiring repeated use and durability, such as reusable stickers, labels, and wearable devices, and for enhancing knot security and facilitates the learning of shoelace tying through color-coded adhesive sections, promoting independence and confidence.

Alternatively, in practice and use, the adhesive composition may be integrated into a layered adhesive tape system including multiple layers of the adhesive, each approximately 0.5 mm thick, allowing for customizable tensile strength and reusability. In this way, the adhesive composition allows for a biodegradable connector including precise control over adhesion strength, unlike hook and loop connectors, which have fixed mechanical properties.

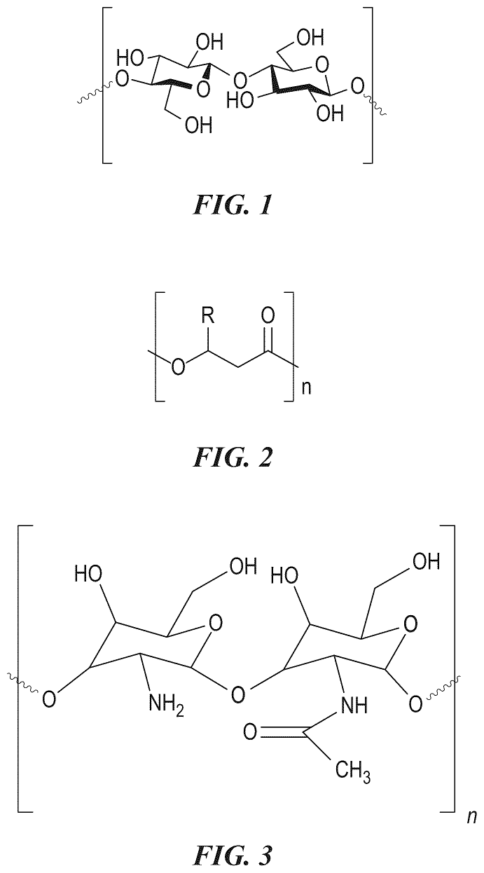

FIG. 1 illustrates the chemical structure of cellulose nanocrystals, showing the repeating β-d-glucopyranose units linked by β(1→4) glycosidic bonds, highlighting the hydroxyl groups available for hydrogen bonding.

FIG. 2 illustrates the chemical structure of polyhydroxyalkanoates, depicting the repeating hydroxyalkanoic acid units linked by ester bonds (-coo-), illustrating the flexible polyester backbone.

FIG. 3 illustrates the chemical structure of chitosan nanoparticles, showing the copolymer of d-glucosamine and n-acetyl-d-glucosamine units linked by β(1→4) glycosidic bonds, emphasizing the presence of amine groups (-nh₂).

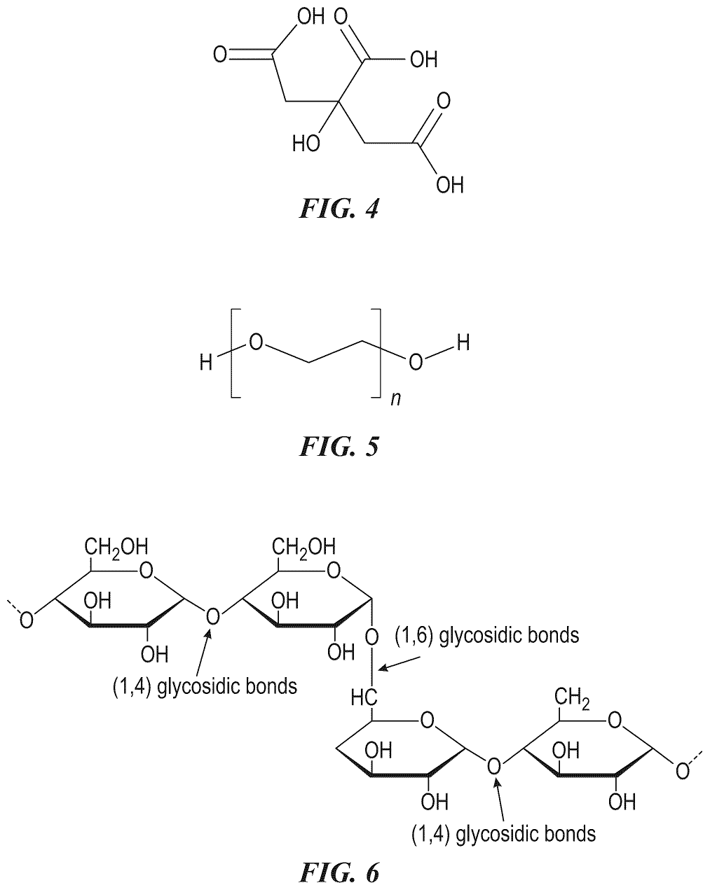

FIG. 4 illustrates the chemical structure of citric acid, displaying the tricarboxylic acid with three carboxyl groups (-cooh) and one hydroxyl group (-oh), indicating potential sites for cross-linking reactions.

FIG. 5 illustrates the chemical structure of polyethylene glycol, illustrating the repeating ethylene oxide units (-ch₂ch₂o-) and terminal hydroxyl groups, contributing to flexibility and hydrophilicity.

FIG. 6 illustrates the chemical structure of modified starch, presenting the amylose and amylopectin units linked by α(1→4) and α(1→6) glycosidic bonds, demonstrating the modification points enhancing viscosity.

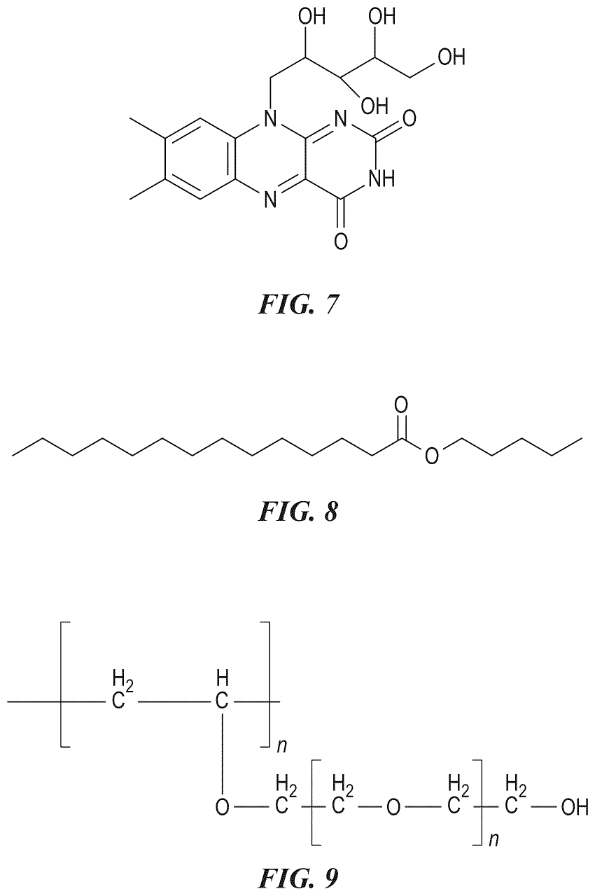

FIG. 7 illustrates the chemical structure of riboflavin (vitamin b₂), showing the isoalloxazine ring connected to a ribitol chain, highlighting the conjugated double bonds responsible for UV absorption.

FIG. 8 illustrates a representative chemical structure of bio-based phase-change materials, such as a fatty acid ester with long hydrocarbon chains, depicting the ester linkage and alkane chains involved in thermal energy storage.

FIG. 9 illustrates the chemical structures of the self-healing polymers, including polyvinyl alcohol and polyethylene glycol blend, showing the vinyl alcohol units and ethylene oxide units, illustrating the hydrogen bonding interactions.

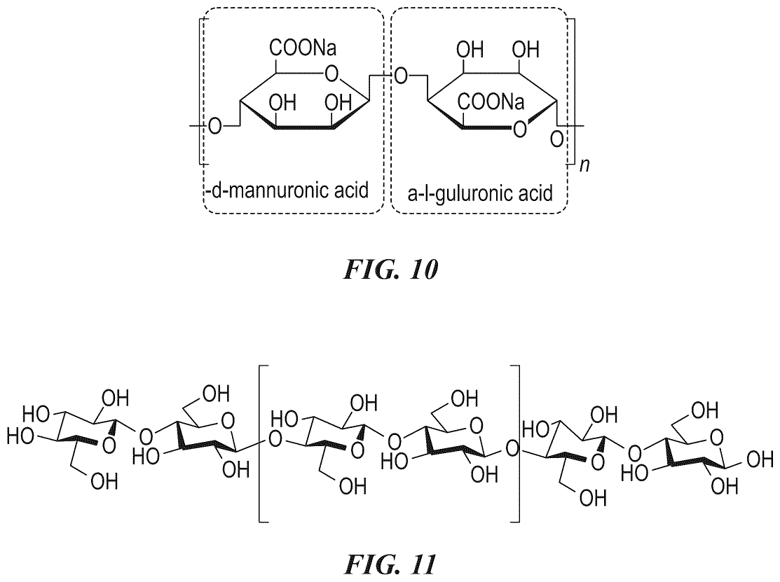

FIG. 10 illustrates the chemical structure of alginate, displaying the alternating β-d-mannuronic acid and α-1-guluronic acid residues linked by β(1→4) glycosidic bonds, indicating sites for ionic cross-linking.

FIG. 11 illustrates the chemical structure of nano-cellulose fibers, similar to CNCs but highlighting the amorphous regions that provide flexibility, with β-d-glucopyranose units linked by β(1→4) glycosidic bonds.

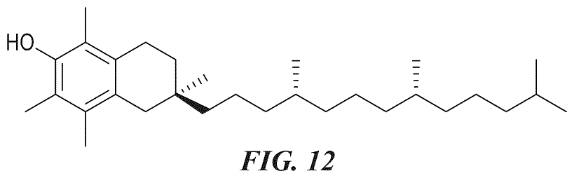

FIG. 12 illustrates the chemical structure of vitamin e (natural antioxidant), featuring the chromanol ring with a phytyl tail, emphasizing the hydroxyl group responsible for antioxidant activity.

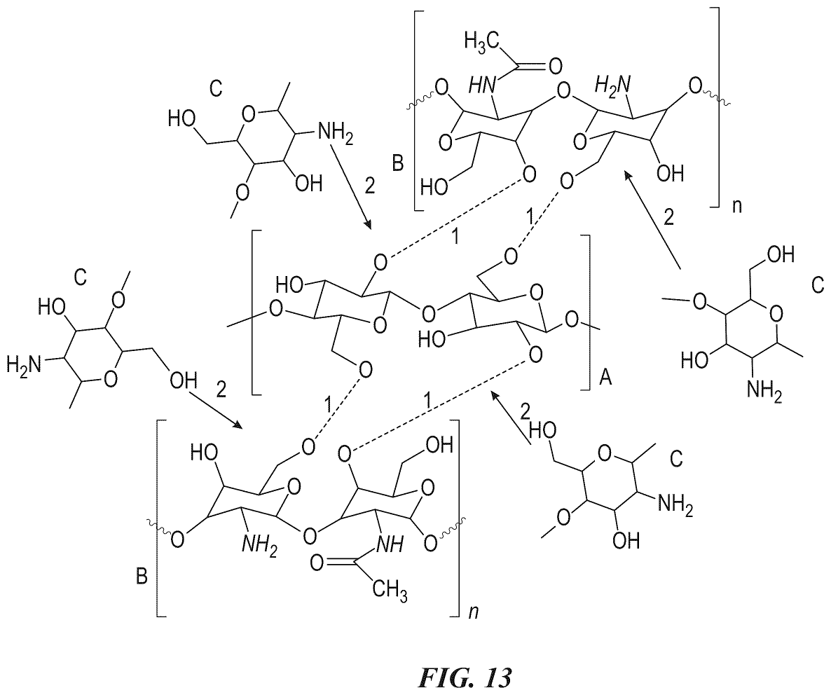

FIG. 13 illustrates the integration of CNCs, NCFs, and chitosan nanoparticles into a matrix, according to some embodiments.

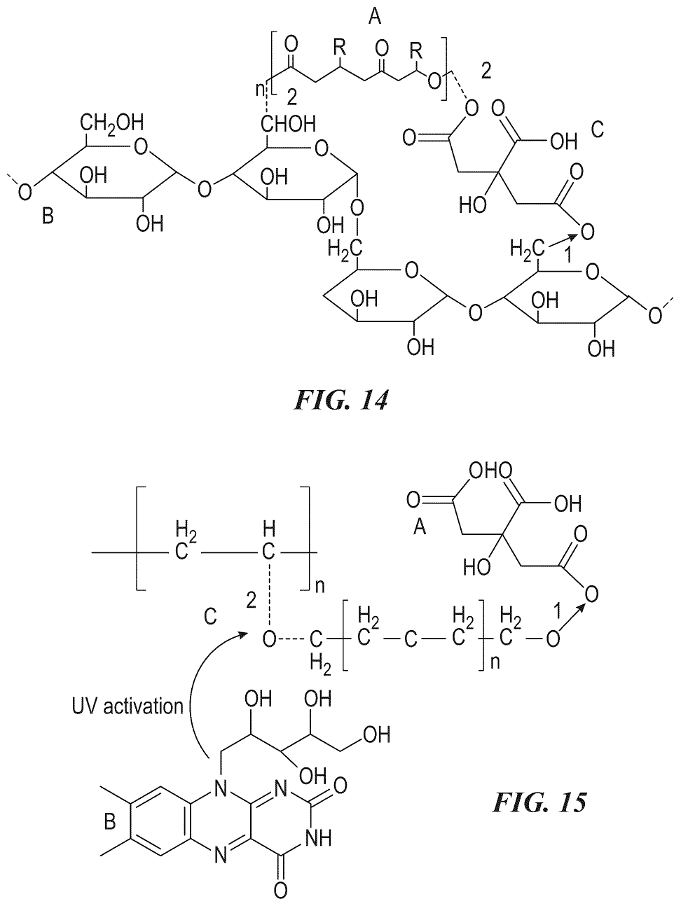

FIG. 14 illustrates biopolymer matrix crosslinking, according to some embodiments.

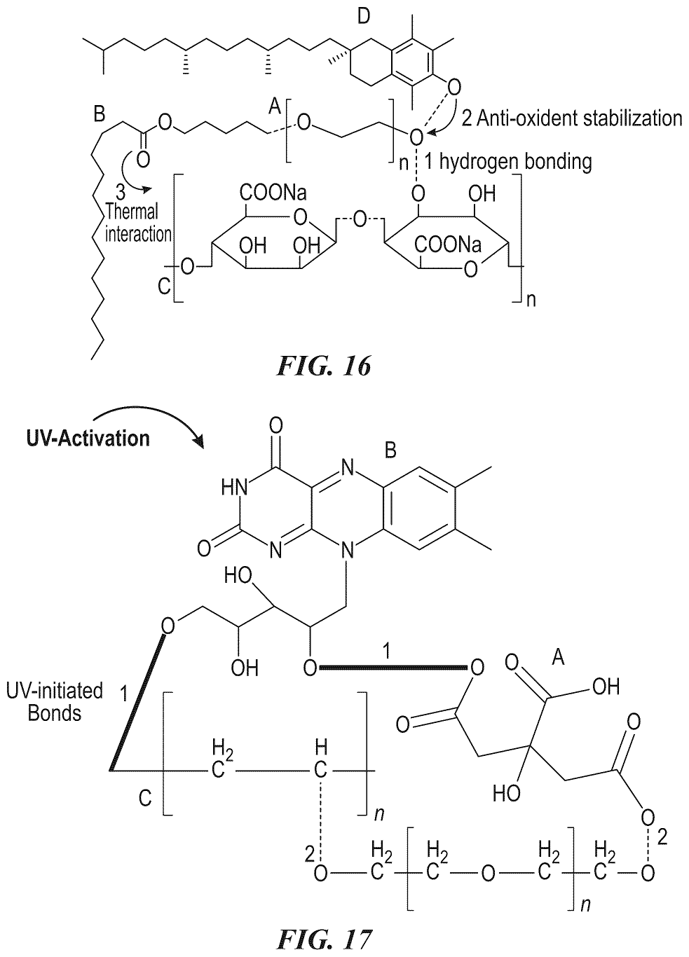

FIG. 15 illustrates crosslinking and curing of the biopolymer matrix, according to some embodiments. FIG. 16 illustrates a method of additive integration, according to some embodiments.

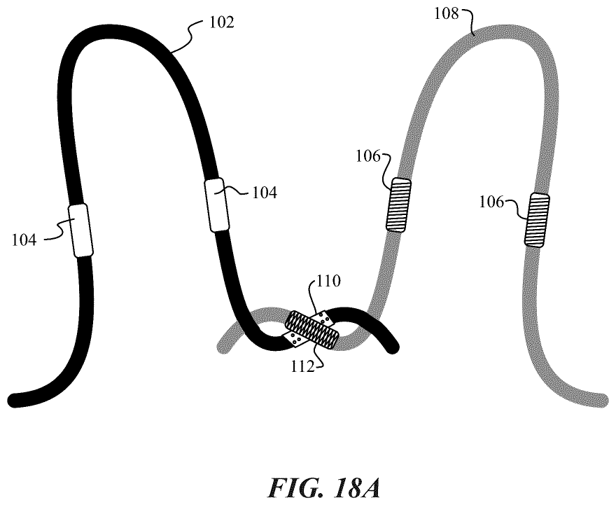

FIG. 17 illustrates a crosslinked polymer matrix, according to some embodiments

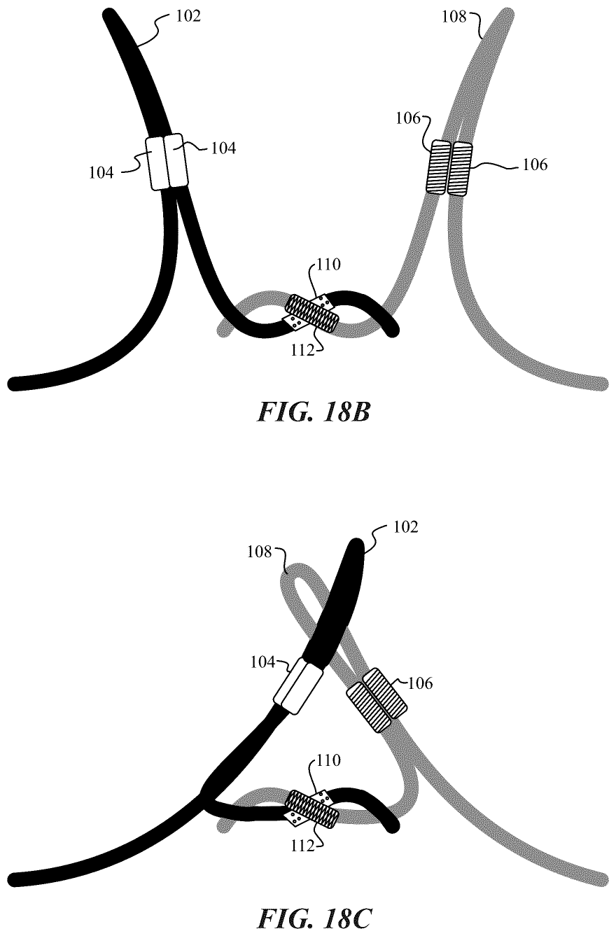

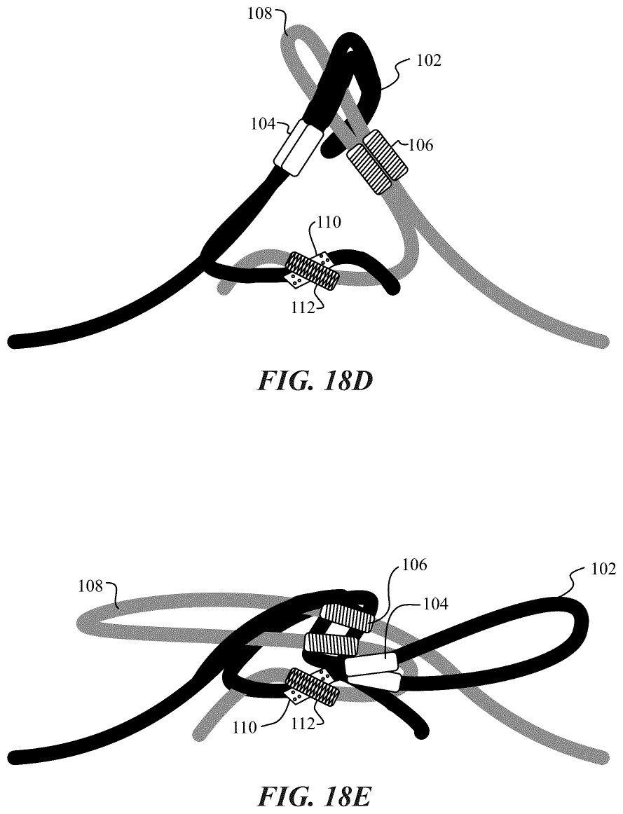

FIGS. 18A-18E illustrate a self-renewing, adhesive-based shoelace system, according to some embodiments. A first lace 102 may include a first set 104 of color coded markers. A second lace 108 may include a second set 106 of color coded markers. A fourth set 110 and fifth set 112 of color coded markers may be included as needed, to facilitate the tying of the first lace 102 and the second lace 108, into, for example, a bow, depicted in FIG. 18E. For example, the first set 104 may be configured to teach a user to pull or group the first set 104 together and the second set 106 together before looping the first lace 102 over the second lace 108, under the second lace 108, and through, to form a bow or know.

In this disclosure, the descriptions of the various embodiments have been presented for purposes of illustration and are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein. Thus, the appended claims should be construed broadly, to include other variants and embodiments, which may be made by those skilled in the art.

It will be appreciated by persons skilled in the art that the present embodiment is not limited to what has been particularly shown and described hereinabove. A variety of modifications and variations are possible considering the above teachings without departing from the following claims.

I/We claim:

1. A one-part, biodegradable, food-safe, and self-renewing adhesive composition, comprising:
about 10% to about 40% by weight of one or more nanomaterial reinforcement agents selected from cellulose nanocrystals (CNCs), nano-cellulose fibers (NCFs), chitosan nanoparticles, or mixtures thereof;
about 5% to about 25% by weight of one or more biopolymer matrix components selected from polyhydroxyalkanoates (PHAs), polyvinyl alcohol (PVA), modified starch, or combinations thereof;
about 5% to about 20% by weight of crosslinking and curing agents, including at least one photoinitiator configured to initiate crosslinking upon exposure to ultraviolet (UV) light;
about 5% to about 25% by weight of additives and enhancers that confer washability, thermal adaptability, self-healing, or antimicrobial properties; and
about 0.1% to about 5% by weight of at least one natural antioxidant,
wherein the adhesive composition is wash-reactivatable, restoring at least 80% of its initial tensile strength after washing and drying, and remains skin-safe and hypoallergenic for applications involving prolonged skin contact.

2. The adhesive composition of claim 1, wherein the nanomaterial reinforcement agents include:
about 19.6% by weight cellulose nanocrystals (CNCs),
about 4.9% by weight nano-cellulose fibers (NCFs), and
about 9.8% by weight chitosan nanoparticles,
collectively enhancing tensile strength and antimicrobial efficacy through hydrogen bonding and ionic interactions.

3. The adhesive composition of claim 1, wherein the chitosan nanoparticles exhibit a degree of deacetylation of at least 85%, maximizing antimicrobial properties and adhesion.

4. The adhesive composition of claim 1, wherein the biopolymer matrix components comprise about 14.71% polyhydroxyalkanoates (PHAs) and about 7.84% modified starch by weight, providing enhanced flexibility, thermal stability, and mechanical strength via hydrogen bonding and ester linkages.

5. The adhesive composition of claim 1, wherein the crosslinking and curing agents include:
about 7.84% citric acid forming ester or amide bonds with hydroxyl or amine-bearing components, and
about 4.9% riboflavin (vitamin B₂) acting as a photoinitiator for UV curing,
thereby achieving an adjustable tensile strength of about 15 psi to about 21,525.9 psi under varying UV intensities.

6. The adhesive composition of claim 1, wherein the additives and enhancers include a polyvinyl alcohol/polyethylene glycol (PVA/PEG) blend, forming a dynamic hydrogen-bonded network that self-heals up to 90% of original mechanical strength after damage or repeated wash cycles.

7. The adhesive composition of claim 1, wherein the additives and enhancers further comprise bio-based phase-change materials (PCMs) selected from fatty acid esters, enabling thermal adaptability in a range of about −40° C. to about 220° C.

8. The adhesive composition of claim 1, wherein the cellulose nanocrystals are surface-modified with carboxyl or amino functional groups, further enhancing intermolecular bonding and overall cohesion within the adhesive matrix.

9. The adhesive composition of claim 1, wherein the tensile strength is modeled by the formula:
$$PSI ( t ) = PSI\_base + ( PSI\_max - PSI\_base ) \times ( 1 - e \hat{} ( - k \times I \times t ) )$$
where:
PSI(t) is the tensile strength at time t.
PSI_base=10 PSI is the base tensile strength before UV curing.
PSI_max=21,525.9 PSI is the maximum achievable tensile strength.
k=0.02 (cm²/W-s) is the rate constant.
I is the UV intensity in W/cm².
t is the exposure time in seconds.
e is Euler's number (approximately 2.71828).
thereby enabling fine-tuned curing and tensile strength based on controllable UV parameters.

10. The adhesive composition of claim 1, wherein the composition retains at least 70% of its initial adhesive strength after 300-350 wash cycles, reactivated by simple washing and drying, thereby reducing waste and extending product life.

11. A shoelace system for enhanced knot security and learning, comprising:
a shoelace with two terminal ends;
one or more adhesive tabs formed from the adhesive composition of claim 1, positioned at strategic points to guide knot formation; and
color-coded indicators associated with said adhesive tabs,
wherein the adhesive tabs are wash-reactivatable, maintaining at least 70% of their adhesive force after 300 wash cycles, and the system is fully biodegradable and skin-safe for repeated consumer use.

12. The shoelace system of claim 11, wherein base adhesive tabs are placed proximate each terminal end to secure an initial knot, and loop adhesive tabs are located at a mid-length portion of the lace, assisting loop formation and reducing knot slippage, thereby aiding motor skill development.

13. A layered tape system, comprising:
a plurality of adhesive layers formed from the adhesive composition of claim 1, each layer having a thickness of about 0.5 mm;
the layers being UV-cured sequentially or simultaneously to achieve customizable tensile strength and self-healing properties; and
wherein the tape system remains adhesive after 300 wash cycles and withstands temperature fluctuations from about −40° C. to about 220° C.

14. The layered tape system of claim 13, wherein the tape is applied to structural components in automotive or aerospace settings, providing biodegradable and high-strength bonding under extreme operating conditions.

15. A resealable packaging system, comprising:
a biodegradable packaging substrate;
a closure mechanism integrated with the adhesive composition of claim 1; and
wherein the adhesive composition enables multiple open-and-close cycles without significant loss of sealing force, such that the packaging retains at least 70% of its initial sealing strength after 20 re-sealing operations and is food-safe, complying with FDA/EFSA regulations.

16. The resealable packaging system of claim 15, wherein the wash-reactivatable property of the adhesive composition allows repeated cleaning and reuse of the packaging, maintaining product freshness and reducing environmental waste.

17. A medical device configured for prolonged skin contact, comprising:
a biocompatible substrate; and
a layer of the adhesive composition of claim 1 applied thereto, wherein the adhesive composition is sterilizable via gamma irradiation, autoclaving, or ethylene oxide, and retains at least 80% of its peel strength after five sterilization cycles, providing hypoallergenic and wash-reactivatable bonding.

18. The medical device of claim 17, wherein the adhesive layer is arranged as a carrier-free film forming a self-healing wound dressing, enabling repeated repositioning on skin without significant loss of adhesion or antimicrobial efficacy.

19. A method of preparing the adhesive composition of claim 1, comprising:
measuring cellulose nanocrystals (CNCs), nano-cellulose fibers (NCFs), chitosan nanoparticles, modified starch, citric acid, and antioxidants according to specified weight percentages;
adding polyhydroxyalkanoates (PHAs), polyethylene glycol (PEG), a self-healing polymer blend, riboflavin (vitamin B₂), bio-based phase-change materials, and alginate or carrageenan as solutions or dispersions;
mixing all components under high-shear conditions for 60-90 minutes at a temperature of about 25° C. to 35° C., controlling viscosity as needed;
optionally subjecting the mixture to vacuum degassing for 15-30 minutes to remove entrapped air; and
storing the composition in airtight, UV-resistant containers at room temperature,
wherein the resulting adhesive composition exhibits adjustable tensile strength from about 15 psi to about 21,525.9 psi upon UV curing and wash-reactivatable properties enabling multiple usage cycles.

20. A method of reactivating the adhesive composition of claim 1, comprising:
applying the adhesive composition to a substrate and curing it under controlled UV intensity to achieve an initial tensile strength;
washing or rinsing the cured adhesive in water; and
drying the adhesive, wherein the adhesive restores at least 80% of its initial adhesive strength upon drying, enabling repeated reactivation without additional curing agents.

21. The method of claim 20, wherein the adhesive is reactivated for at least 10 cycles of wash-and-dry without losing more than 20% of its original peel strength.

22. The adhesive composition of claim 1, wherein esterification occurs between carboxyl groups of citric acid and hydroxyl groups on cellulose nanocrystals, forming a covalently crosslinked network that boosts tensile strength to at least 21,500 psi.

23. The adhesive composition of claim 1, wherein amine groups on chitosan nanoparticles form amide bonds with carboxyl groups of citric acid, further improving antimicrobial efficacy and film integrity.

24. The adhesive composition of claim 4, wherein the ratio of PHAs to modified starch is between 2:1 and 3:1, optimizing flexibility and viscosity for footwear applications.

25. The adhesive composition of claim 6, wherein the PVA/PEG blend achieves 90% mechanical recovery within 30 minutes at ambient temperature after a tensile or shear failure event.
